(19) United States
(12) Patent Application Publication
(10) Pub. No.: US 2025/0152168 A1
(43) Pub. Date: May 15, 2025

(54) COMPRESSION RETAINERS FOR STAPLE CARTRIDGES WITH IMPLANT ADJUNCTS

(71) Applicant: CILAG GMBH INTERNATIONAL, ZUG (CH)

(72) Inventors: MARK ZEINER, LOVELAND, OH (US); SARAH ALEXANDRA SCULLY, CINCINNATI, OH (US); MICHAEL J. VENDELY, LEBANON, OH (US); MIAH IGWE, CINCINNATI, OH (US); BRIAN ROUNTREE, MAINEVILLE, OH (US)

(21) Appl. No.: 18/505,647

(22) Filed: Nov. 9, 2023

(51) Int. Cl. A61B 17/072

(52) U.S. Cl. CPC A61B 17/072; A61B 2017/07271

(57) ABSTRACT

Retainers and retainer systems for staple cartridges with implantable adjuncts are disclosed. The retainers and retainer systems enable a compressive force to be applied to implantable adjuncts on staple cartridges.

### FIELD OF INVENTION

The present disclosure generally relates to retainers and retainer systems for staple cartridges with implantable adjuncts. More specifically, the present disclosure relates to retainers and retainer systems that enable a compressive force to be applied to implantable adjuncts on staple cartridges.

### BACKGROUND

Stapling is a crucial aspect of many surgical procedures, such as gastrointestinal, thoracic, and gynecological surgeries. Staple cartridges used in said stapling procedures may include an implantable adjunct on the deck of the cartridge. Care must be taken to ensure the implantable adjunct is properly adhered to the deck so that it is not dislodged from the deck during shipment or, importantly, during surgery before the adjunct is positioned at the treatment site.

### SUMMARY

It is an object of the present designs to provide devices and methods to meet the above-stated needs. The designs can be for systems and devices for protecting an implantable adjunct on a staple cartridge, while also providing structure to allow compression of the adjunct to the deck of the staple cartridge before being used in surgery.

The instant disclosure describes a staple cartridge. The staple cartridge includes an elongate body. The elongate body includes a deck. The elongate body defines a plurality of staple pockets, each of the staple pockets accessible via an opening in the deck. The staple cartridge includes an implantable adjunct removably secured to the deck. The staple cartridge includes a retainer removably securable to the elongate body. The retainer includes a first retainer tab extending laterally with respect to a longitudinal axis of the retainer. The retainer includes a second retainer tab extending laterally with respect to the longitudinal axis of the retainer. The retainer includes the first retainer tab and the second retainer tab movable from a first position to a second position. In the second position, the retainer provides a force to compress the implantable adjunct against the deck of the elongate body. And at least a portion of the first retainer tab and the second retainer tab is closer to the elongate body in the second position than in the first position.

The instant disclosure describes a method of loading a staple cartridge onto an end effector. The method includes inserting a staple cartridge into a channel of a first jaw frame of an end effector. The method includes moving a first retainer tab and a second retainer tab of a retainer from a first position to a second position, thereby causing a force to be applied to an implantable adjunct on a deck of the staple cartridge. The force increases an adhesion of the implantable adjunct to the deck by providing pressure to an attachment material disposed between the implantable adjunct and the deck. The method includes removing the retainer from the staple cartridge.

The instant disclosure describes a staple cartridge. The staple cartridge includes an elongate body. The elongate body includes a deck and defines a plurality of staple pockets. Each of the staple pockets is via an opening in the deck. The staple cartridge includes an implantable adjunct removably secured to the deck. The staple cartridge includes a retainer removably securable to the elongate body. The retainer includes a retainer body comprising a first lateral wall and a second lateral wall. The retainer includes a film extending between the first lateral wall and the second lateral wall and disposed along a side of the implantable adjunct opposite the deck.

The instant disclosure describes a method of loading a staple cartridge onto an end effector. The method includes inserting a staple cartridge into a channel of a first jaw frame of an end effector, the staple cartridge being at least partially enclosed within a retainer. The method includes clamping an anvil of an end effector onto a film of the retainer. The method includes tearing the film with the anvil to create a torn portion. The method includes pressing, with the anvil, the torn portion onto an implantable adjunct on a deck of the staple cartridge. The method includes removing the retainer from the staple cartridge.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a perspective view of a replaceable staple cartridge without an adjunct.

FIG. 2A is a schematic showing a replaceable staple cartridge, an attachment material, and an implantable adjunct, according to aspects of the present disclosure.

FIG. 2B is a schematic showing the replaceable staple cartridge, attachment material, and implantable adjunct of FIG. 2A assembled, according to aspects of the present disclosure.

FIG. 2C is a perspective view of the assembled staple cartridge, attachment material, and implantable adjunct shown in FIG. 2B, according to aspects of the present disclosure.

FIG. 3A is a side-view schematic of a staple cartridge being loaded into a surgical instrument, according to aspects of the present disclosure.

FIG. 3B is a schematic of an implantable adjunct stapled to tissue, according to aspects of the present disclosure.

FIG. 4A is a perspective view of an example staple cartridge retainer with foldable retainer tabs, according to aspects of the present disclosure.

FIG. 4B is a perspective view of the example staple cartridge retainer shown in FIG. 4A and shows the retainer tabs folded into a second position closer to the cartridge body, according to aspects of the present disclosure.

FIG. 5A is a perspective view of an example breakaway staple cartridge retainer with foldable retainer tabs, according to aspects of the present disclosure.

FIG. 5B is a perspective view of the example staple cartridge retainer shown in FIG. 5A loaded into an end effector, according to aspects of the present disclosure.

FIG. 5C is a perspective view of the example staple cartridge retainer shown in FIGS. 5A and 5B and shows the retainer tabs folded into a second position closer to the cartridge body, according to aspects of the present disclosure.

FIG. 5D is a perspective view of the example staple cartridge retainer shown in FIGS. 5A-5C and shows two breakaway portions separated, according to aspects of the present disclosure.

FIG. 5E is a bottom perspective view of the example staple cartridge retainer shown in FIGS. 5A-5D and shows two breakaway portions separated, according to aspects of the present disclosure.

FIG. 5F is a detailed view of the example staple cartridge retainer shown in FIGS. 5A-5E and shows the retainer tabs folded into a second position closer to the cartridge body, according to aspects of the present disclosure.

FIG. 6A is a perspective view of an example breakaway staple cartridge retainer with foldable retainer tabs and a separate adjunct cap, according to aspects of the present disclosure.

FIG. 6B is a perspective view of the example staple cartridge retainer shown in FIG. 6A loaded into an end effector and the retainer tabs folded, according to aspects of the present disclosure.

FIG. 6C is a perspective view of the example staple cartridge retainer shown in FIGS. 6A and 6B and shows two breakaway portions separated, according to aspects of the present disclosure.

FIG. 6D is a perspective view of the separate adjunct cap shown in FIGS. 6A-6C and with the retainer removed from said adjunct cap, according to aspects of the present disclosure.

FIG. 6E is a bottom perspective view of the example staple cartridge retainer shown in FIGS. 6A-6C, according to aspects of the present disclosure.

FIG. 7A is a perspective view of an example staple cartridge retainer with retainer tabs pivotable with respect to each other, according to aspects of the present disclosure.

FIG. 7B is an end view of the example staple cartridge retainer shown in FIG. 7A and shows the retainer tabs in an initial, uncompressed configuration, according to aspects of the present disclosure.

FIG. 8A is a perspective view of an example retainer having a film, according to aspects of the present disclosure.

FIG. 8B is a perspective view of the example retainer shown in FIG. 8A loaded into a staple cartridge, according to aspects of the present disclosure.

FIG. 8C is a perspective view of the example retainer shown in FIGS. 8A and 8B loaded

FIG. 8D is a perspective view of the example retainer shown in FIGS. 8A-8C loaded into an end effector of a surgical instrument and wherein an anvil of the end effector has torn the film, according to aspects of the present disclosure.

FIG. 9 is a flowchart for an example method of loading a staple cartridge onto an end effector, according to aspects of the present disclosure.

FIG. 10 is a flowchart for an example method of loading a staple cartridge onto an end effector, according to aspects of the present disclosure.

### DETAILED DESCRIPTION

Specific examples of the present invention are now described in detail with reference to the Figures, where identical reference numbers indicate elements which are functionally similar or identical. The examples provide solutions for staple cartridge systems that include an implantable adjunct. An implantable adjunct can be used in stapling surgery to account for differing tissue thicknesses across the length of the stapling surface. For instance, a length of tissue clamped in an end effector of a surgical instrument may by thicker at one end of the staple cartridge that at the other end. However, the staple cartridge may be loaded with staples of a single length, meaning the staples may be properly sized for the thicker section of tissue, but may be too long for the thinner section of tissue. If the staples are too long, proper compression of the tissue at the staple site may be compromised, leading to undesired bleeding. An implantable adjunct can account for this differing tissue thickness by providing support for the thinner sections of tissue. Where the tissue is thick, the implantable adjunct can be compressed all the way down since no additional thickness is needed to account for the staple length. Where the tissue is thin, the implantable adjunct is not as compressed, meaning the adjunct provides the additional thickness needed to account for the staple length, thereby providing proper compression in that section of the tissue.

The implantable adjunct must be properly, yet reversibly, adhered to the deck of the staple cartridge so that it does not become dislodged during shipment or, importantly, during the surgical procedure. For instance, during surgery the staple cartridge is loaded into an end effector of a cutter/stapler surgical instrument, sent through a cannula to a surgical site, traversed through and around tissue, and then positioned at the target tissue site that will be cut and stapled by the surgical instrument. If the implantable adjunct is not properly adhered to the deck of the staple cartridge, it may become dislodged from the deck during this procedure. The adjunct is adhered to the cartridge deck with an attachment material, which needs to be sticky or tacky enough to keep the adjunct adhered to the deck, but not so sticky that it is difficult to detach from the deck after the stapling procedure is completed. As such, the present retainer systems provide solutions that ensure the adjunct is properly adhered to the staple cartridge right before the staple cartridge is positioned at the treatment site. These systems provide such solutions by incorporating retainers and retainer systems that enable a temporary compressive force to be applied to implantable adjuncts on staple cartridges before they are used in surgery.

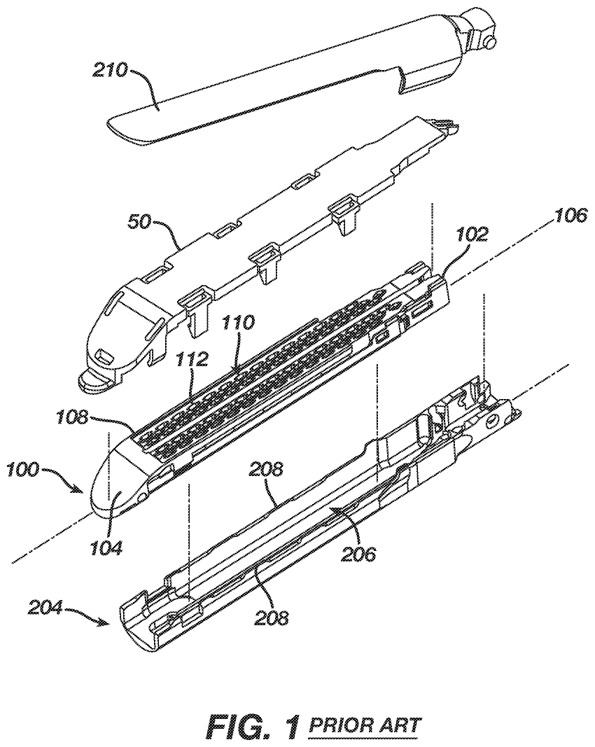

FIG. 1 provides background on how the presently described retainer systems improve upon prior designs because the current systems take into account an adjunct being positioned on the staple cartridge. Referring now to the figure, FIG. 1 shows an exploded view of a prior staple cartridge 100 that does not include an implantable adjunct on deck 108 thereof. In these prior examples, retainer 50 can be attached to staple cartridge 100 from proximal end 102 to distal end 104 to ensure that staples within various staple pockets 110 do not fall out of openings 112 within deck 108. Retainer 50, therefore, is merely a static device with a function of preventing staples from falling out before staple cartridge 100 is positioned within channel 206 of first jaw frame 204 of end effector 202. Retainer 50 is simply removed when staple cartridge 100 is inserted between channel rails 208 of channel 206.

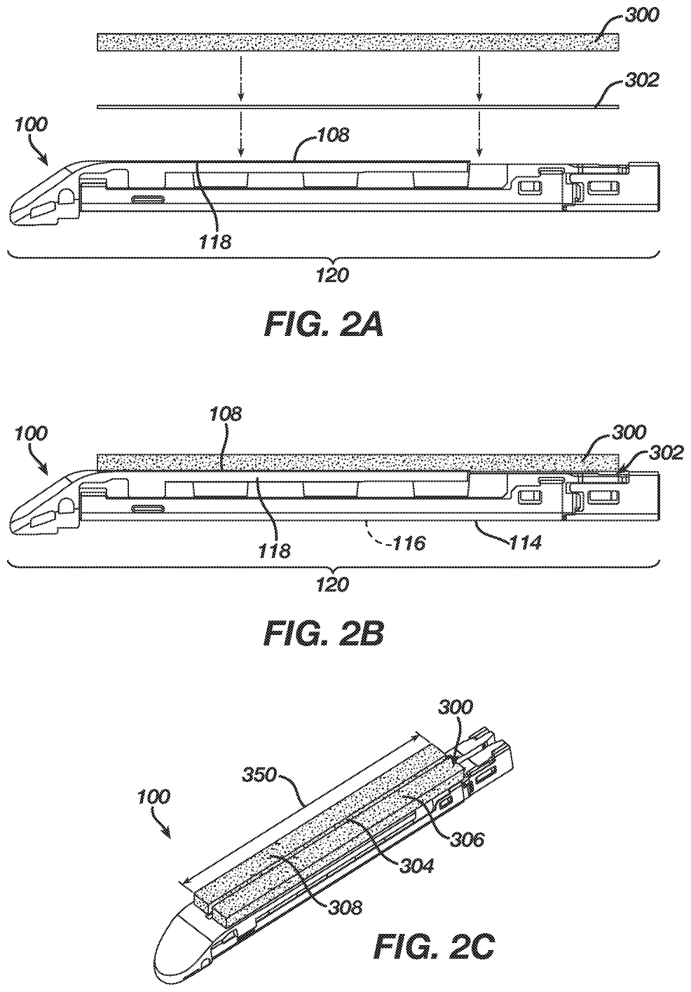

FIGS. 2A-2C illustrate staple cartridges that include an implantable adjunct. As seen in the exploded view of FIG. 2A, the system includes staple cartridge 100 (which is substantially similar to the staple cartridge shown in FIG. 1) and implantable adjunct 300 which is adhered to deck 108 that is positioned along elongate body 120 of staple cartridge 100. Adjunct 300 can be adhered to staple cartridge 100 with attachment material 302. As described above, attachment material 302 can provide sufficient adhesion for adjunct 300 to remain adhered to deck 108 when being positioned at the treatment site, but the adhesion does not impair the ability of adjunct 300 from being detached from deck 108 when being implanted. In some instances, attachment material can be an adhesive, adhesive strip, double-sided tape, and the like. FIG. 2B shows adjunct 300 adhered to deck 108 via attachment material 302. FIG. 2C is a perspective view of adjunct 300 adhered to deck 108. For background, the staples of the systems described herein are fired through adjunct 300 during the stapling procedure. In some instances, adjunct 300 can include sled groove 304 within length 350 of the adjunct. Sled groove 304 provides a path for a knife (not shown in figures) to traverse such that the knife does not need to cut through adjunct 300, thereby preserving the edge on the knife. When adjunct 300 includes sled groove 304, adjunct 300 can be considered to be separated into adjunct first side 306 and adjunct second side 308. In some examples, adjunct 300 can include laminated layers, such as a foam and/or porous material laminated with a mesh material, wherein the sled groove 304 is disposed in the foam and/or porous material but the mesh material remains intact. In other examples, adjunct 300 can include a film layer and/or a mesh layer. The film layer can comprise material commonly used with absorbable monofilament sutures and can be heat processed with a mesh layer to act as a bonding agent to hold the mesh and foam of the adjunct 300 together.

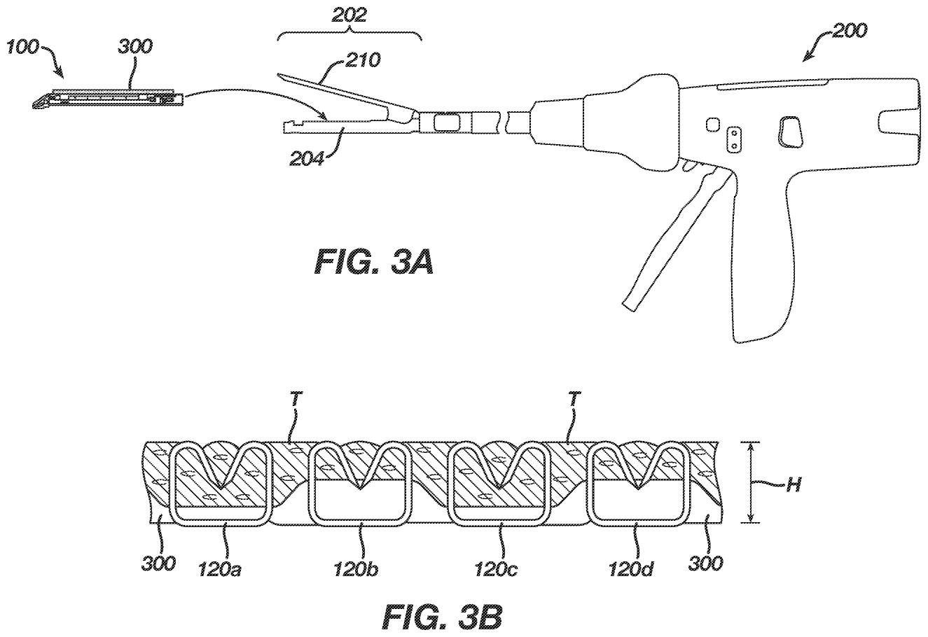

FIG. 3A is a side-view schematic of staple cartridge 100 being loaded into a surgical instrument, i.e., surgical instrument 200. Staple cartridge 100 is loaded into end effector 202 before being positioned at the treatment site. As described above, staple cartridge 100 is inserted into first jaw frame 204. Anvil 210 clamps down toward staple cartridge 100 during the stapling procedure. Once the tissue is stapled, anvil 210 opens to leave the staples and adjunct attached to the tissue. Staple cartridge 100 remains in first jaw frame 204 as surgical instrument 200 is removed from the treatment site. Although FIG. 3A shows staple cartridge 100 without a retainer attached thereto, the example retainers (i.e., retainer 400) described herein can be inserted into first jaw frame 204 while attached to said staple cartridge 100. As stated above, implantable adjunct 300 can account for this differing tissue thickness by providing support for the thinner sections of tissue. Where the tissue is thick, implantable adjunct 300 can be compressed all the way down since no additional thickness is needed to account for the staple length. Where the tissue is thin, the implantable adjunct 300 is not as compressed, meaning the adjunct provides the additional thickness needed to account for the staple length, thereby providing proper compression in that section of the tissue. FIG. 3B is a schematic showing the implantable adjunct 300 stapled to tissue (T) having different thickness. The individual staples 120a,b,c,d have the same height (H), so the implantable adjunct 300 fills in the space for thinner sections of tissue (i.e., the tissue (T) shown at staples 120b and 120d). For thicker sections of tissue (i.e., the tissue (T) shown at staples 120a and 120c), the implantable adjunct 300 is more compressed as the staples do not need the additional space (i.e., height) filled in by the implantable adjunct 300.

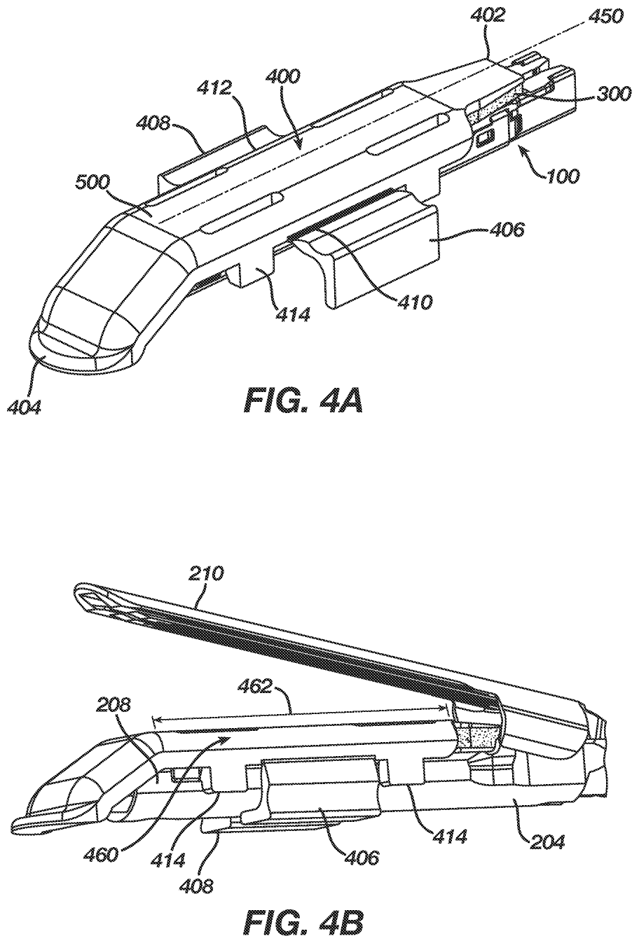

Referring now to FIGS. 4A and 4B, the example implementation shows a staple cartridge and retainer system that can protect an adjunct during shipment of the staple cartridge and also provide a compressive force to the adjunct. Retainer 400 includes first retainer tab 406 and second retainer tab 408 disposed between proximal end 402 and distal end 404 of retainer 400. Retainer 400 includes retainer body 500 extending along longitudinal axis 450 of retainer 400. Retainer body 500 provides protection of adjunct 300, which is disposed along deck 108 of staple cartridge 100 (see FIGS. 2A-2C for views of staple cartridge 100). First retainer tab 406 and second retainer tab 408 are connected to retainer body 500 via first living hinge 410 and second living hinge 412, respectively. In certain implementations, staple cartridge 100 can be shipped with retainer 400 attached thereto, such that retainer 400 protects adjunct 300 from being dislodged during shipment. First retainer tab 406 and second retainer tab 408 can be shipped in an initial, unfolded state as shown in FIG. 4A.

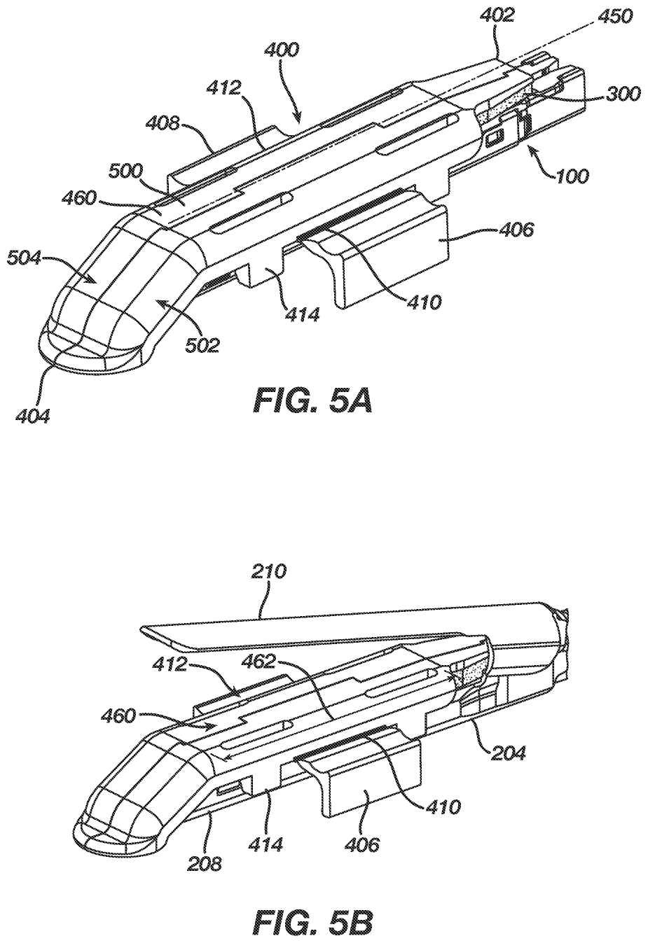

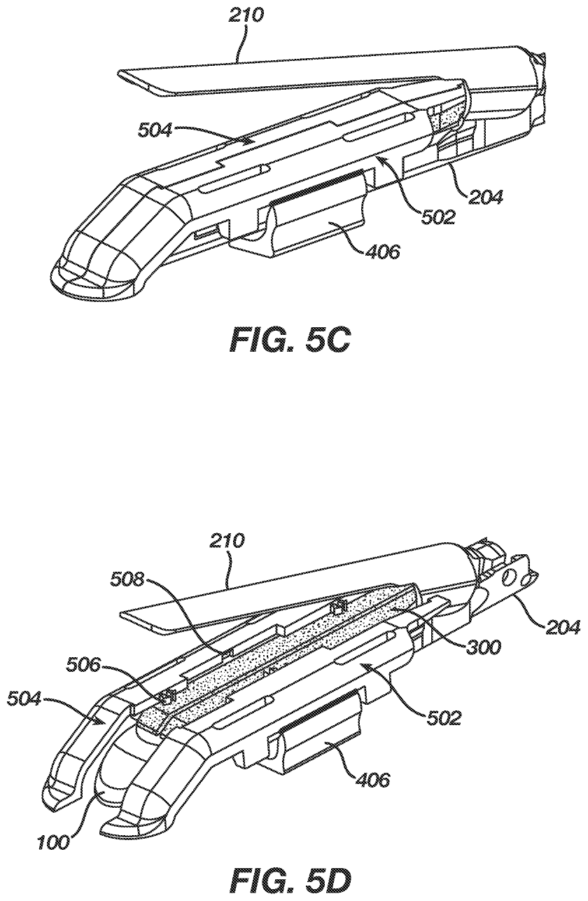

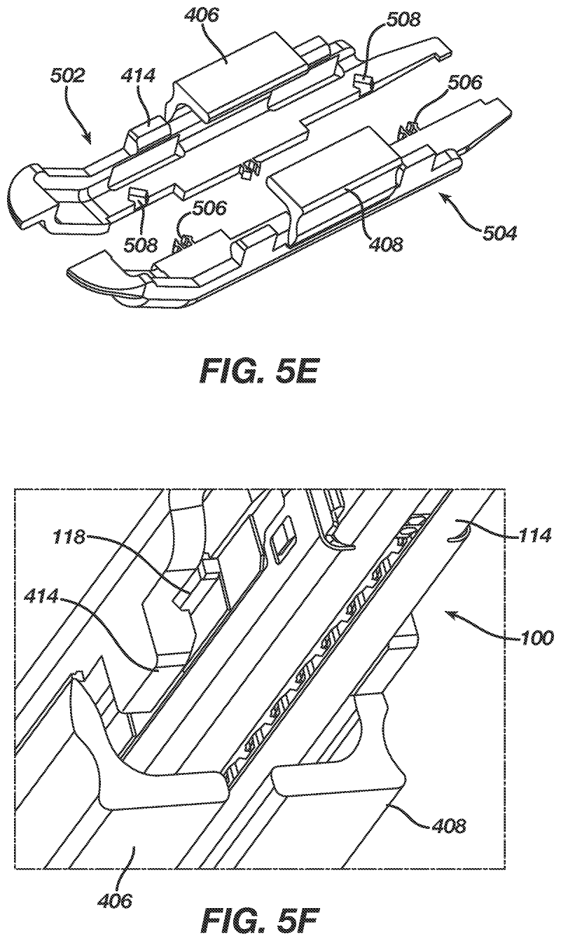

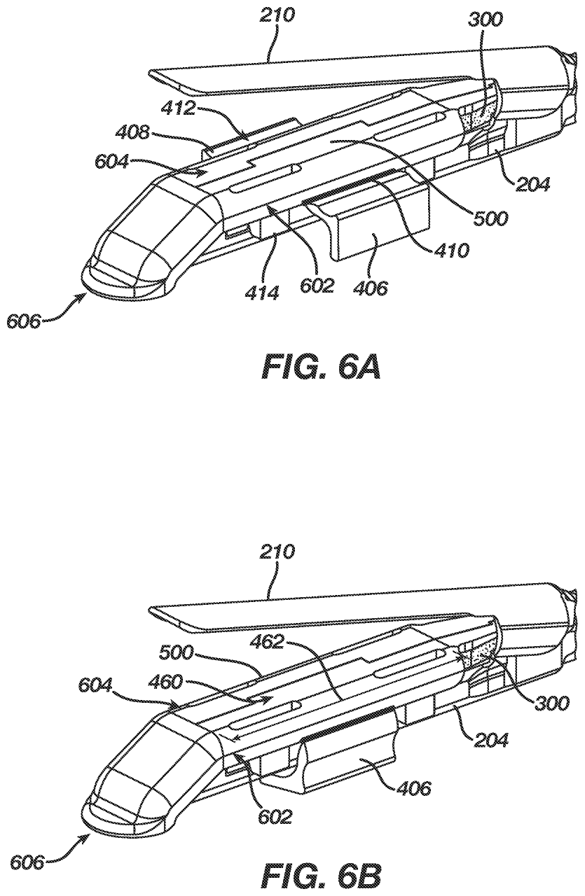

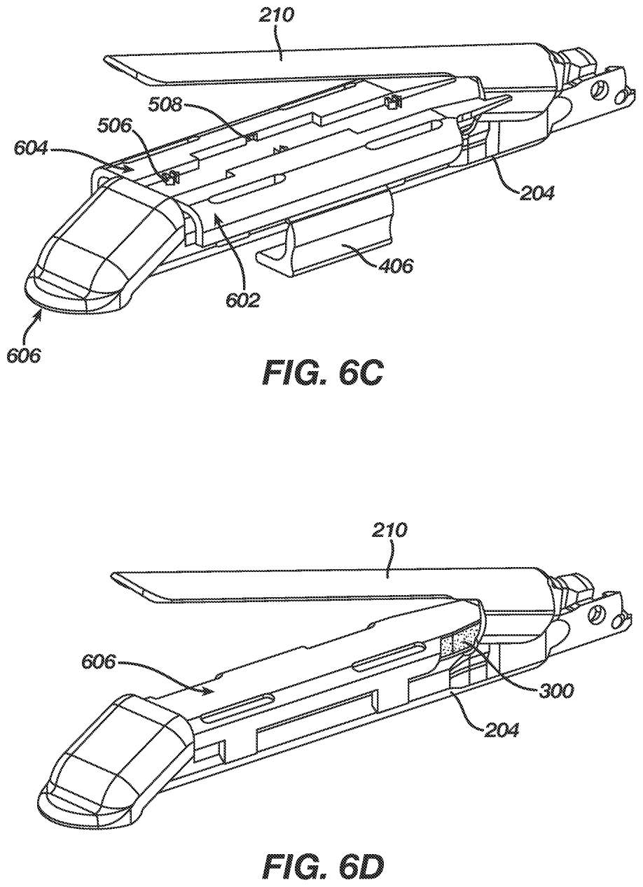

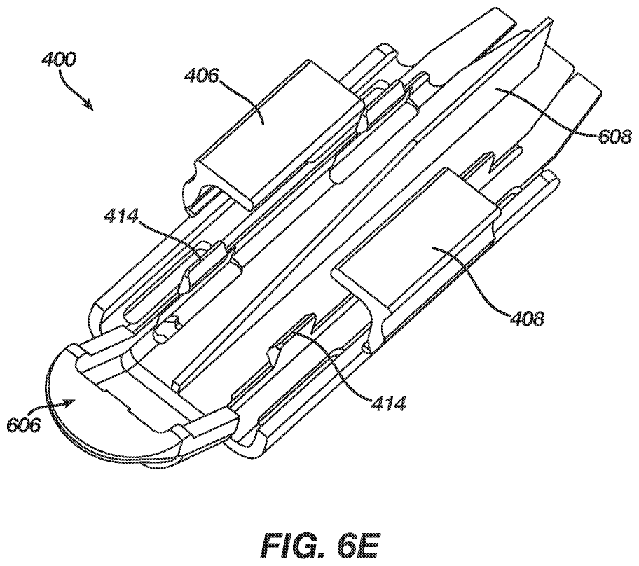

To provide the beneficial compressive force to adjunct 300, first retainer tab 406 and second retainer tab 408 can be folded from their initial, unfolded position (FIG. 4A) to their folded position (FIG. 4B). As such, first retainer tab 406 and second retainer tab 408 are movable from a first position to a second position, the second position being the folded position. In the second, folded position, retainer 400 provides a force to compress implantable adjunct 300 against the deck 108 of the elongate body 120. At least a portion of the first retainer tab 406 and the second retainer tab 408 are closer to the elongate body 120 in the second position than in the first position (compare FIG. 4A to 4B, FIG. 5B to 5C, and FIG. 6A to 6B).

First retainer tab 406 and second retainer tab 408 include angled profiles, as shown in FIGS. 4A and 4B, that allow (i) a portion of the respective tabs 406, 408 to be parallel to the sides of the elongate body 120 when in their folded state, and (ii) a portion of the respective tabs 406, 408 to be positioned on a side of elongate body 120 opposite adjunct 300 once folded into their folded state, i.e. under elongate body 120 (FIG. 4B). By folding and hooking under elongate body 120, retainer body 500 pushes down on adjunct 300, compressing adjunct 300 and attachment material 302 (FIG. 2A) for improved adhesion during the cutting procedure. In some implementations, first retainer tab 406 and second retainer tab 408 can be folded under bottom section 116 of staple cartridge 100 (see FIG. 2A), for example below pan 114 that provides support to elongate body 120. As such, the compression can be applied directly to deck 108 and prior to inserting the cartridge into first jaw frame 204 (see FIG. 3). In other implementations, staple cartridge 100 can be loaded into first jaw frame 204 with retainer 400 attached, and first retainer tab 406 and second retainer tab 408 can be folded under first jaw frame 204 to create the compressive force for adjunct 300. Once compressed, retainer 400 can be removed from staple cartridge 100 to proceed with the stapling procedure.

Retainer 400 includes adjunct shelf 460 along length 462 of retainer 400, the shelf 460 being a substantially planar section of retainer 400. First retainer tab 406 and second retainer tab 408 are positioned centrally along length 462 of adjunct shelf 460 to provide uniform compression along length 350 of implantable adjunct 300. Retainer 400 shown in FIGS. 4A and 4B also includes two retention tabs 414 extending from retainer 400. Retention tabs 414 are outwardly deflectable upon seating into first jaw frame 204. Retention tabs 414 can keep retainer 400 connected to staple cartridge 100 until the cartridge/retainer is inserted into first jaw frame 204. For instance, retention tabs 414 can hook onto cartridge rails 118 of staple cartridge 100 (see FIGS. 2A and 2B). Upon insertion into first jaw frame 204, channel rails 208 of first jaw frame 204 will slip between retention tabs 414 and staple cartridge 100 to deflect them outwardly, thereby releasing them from cartridge rails 118 such that retainer 400 can be removed from staple cartridge 100. FIG. 5F, although depicting a different embodiment of retainer 400, provides an illustration of retention tabs 414 hooked to cartridge rails 118 before being deflected by channel rails 208 of first jaw frame 204.

Referring now to FIGS. 5A-5F, the example shows a staple cartridge and retainer system that can protect an adjunct during shipment of the staple cartridge and also provide a compressive force to the implantable adjunct. Retainer 400 in FIGS. 5A-5F is substantially similar to the retainer described with respect to FIGS. 4A and 4B, but in this implementation retainer 400 includes two different breakaway portions, first breakaway portion 502 and second breakaway portion 504. FIG. 5A shows retainer 400 connected to staple cartridge 100 before being inserted into first jaw frame 204 (see FIG. 3), and FIG. 5B shows retainer 400 and staple cartridge 100 inserted into first jaw frame 204.

Like described above with reference to FIGS. 4A and 4B, retainer 400 in FIGS. 5A-5F includes first retainer tab 406 and second retainer tab 408 and retainer body 500 extending along longitudinal axis 450 of retainer 400. Retainer body 500 provides protection of adjunct 300, as adjunct 300 is disposed along deck 108 of staple cartridge 100 (see FIGS. 2A-2C for views of staple cartridge 100). First retainer tab 406 is connected to first breakaway portion 502 via first living hinge 410, and second retainer tab 408 is connected to second breakaway portion 504 via second living hinge 412. In certain implementations, staple cartridge 100 can be shipped with retainer 400 attached thereto, such that retainer 400 protects adjunct 300 from being dislodged during shipment. As such, first retainer tab 406 and second retainer tab 408 can be shipped in an initial, unfolded state as shown in FIG. 5B. To provide the beneficial compressive force to adjunct 300, first retainer tab 406 and second retainer tab 408 can be folded from their initial, unfolded state (FIG. 5B) to their folded state (FIG. 5C). As described above, in some implementations first retainer tab 406 and second retainer tab 408 can be folded under bottom section 116 of staple cartridge 100 (see FIG. 2A), for example below a pan 114 that provides support to elongate body 120. In other implementations, staple cartridge 100 can be loaded into first jaw frame 204 with retainer 400 attached, and first retainer tab 406 and second retainer tab 408 can be folded under first jaw frame 204 to create the compressive force for adjunct 300. Once compressed, retainer 400 can be removed from staple cartridge 100 to proceed with the stapling procedure. In the example of retainer 400 shown in FIGS. 5A-5F, once the respective retainer tabs 406, 408 are positioned on a side of elongate body 120 opposite implantable adjunct 300, the downward pressure also causes retainer body 500 to break along longitudinal axis 450 such that first breakaway portion 502 and second breakaway portion 504. Separation of first breakaway portion 502 and second breakaway portion 504 can enable retainer 400 to be easily removed from staple cartridge 100 (FIG. 5D).

Referring now specifically to separation of first breakaway portion 502 and second breakaway portion 504, and as shown in FIG. 5E, first breakaway portion 502 or second breakaway portion 504 includes male connector 506 that engages with female connector 508 on the opposite breakaway portion. The male connector 506 in the example embodiment includes two tabs that can be compressed inwardly to snap into the respective female connector 508. As will be appreciated, the breakaway portions can include more than one male connector 506 and female connector 508, spaced evenly along retainer (e.g., along length 462 of adjunct shelf 460). Folding first retainer tab 406 and second retainer tab 408 into their folded states can provide the force to separate the respective male connectors 506 and female connectors 508 to remove retainer 400 from staple cartridge 100 (see FIG. 5D).

Referring to FIG. 5F, retainer 400 shown in the figure also includes two retention tabs 414 extending from retainer 400. Retention tabs 414 are outwardly deflectable upon seating into first jaw frame 204. Retention tabs 414 can keep retainer 400 connected to staple cartridge 100 until the cartridge/retainer is inserted into first jaw frame 204. For instance, retention tabs 414 can hook onto cartridge rails 118 of staple cartridge 100. Upon insertion into first jaw frame 204, channel rails 208 of first jaw frame 204 will slip between retention tabs 414 and staple cartridge 100 to deflect them outwardly, thereby releasing them from cartridge rails 118 such that retainer 400 can be removed from staple cartridge 100.

Referring now to FIGS. 6A-6E, the example shows a staple cartridge and retainer system that can protect an adjunct during shipment of the staple cartridge and also provide a compressive force to the adjunct. Retainer 400 in FIGS. 6A-6E is substantially similar to the retainer described with respect to FIGS. 5A-5F, including having both first breakaway portion 602 and second breakaway portion 604. In this implementation, first breakaway portion 602 and second breakaway portion 604 are positioned upon a separate adjunct cap 606. Like described above with reference to FIGS. 5A-5F, retainer 400 in FIGS. 6A-6E includes first retainer tab 406 and second retainer tab 408. The combination of adjunct cap 606, first breakaway portion 602, and second breakaway portion 604 provides protection of adjunct 300, as adjunct 300 is disposed along deck 108 of staple cartridge 100 (see FIGS. 2A-2C for views of staple cartridge 100) below adjunct cap 606. First retainer tab 406 is connected to first breakaway portion 602 via first living hinge 410, and second retainer tab 408 is connected to second breakaway portion 504 via second living hinge 412. In certain implementations, staple cartridge 100 can be shipped with retainer 400 attached thereto, such that retainer 400 protects adjunct 300 from being dislodged during shipment. As such, first retainer tab 406 and second retainer tab 408 can be shipped in an initial, unfolded state as shown in FIG. 6A. To provide the beneficial compressive force to adjunct 300, first retainer tab 406 and second retainer tab 408 can be folded from their initial, unfolded state (FIG. 6A) to their folded state (FIG. 6B). As described above, in some implementations first retainer tab 406 and second retainer tab 408 can be folded under bottom section 116 of staple cartridge 100 (see FIG. 2A), for example below a pan 114 that provides support to elongate body 120. In other implementations, staple cartridge 100 can be loaded into first jaw frame 204 with retainer 400 attached, and first retainer tab 406 and second retainer tab 408 can be folded under first jaw frame 204 to create the compressive force for adjunct 300.

Referring now to FIGS. 6C and 6D, once the respective retainer tabs 406, 408 are positioned on a side of elongate body 120 opposite implantable adjunct 300, the downward pressure also causes first breakaway portion 602 to separate from second breakaway portion 604. Once the two breakaway portions are separated, the adjunct cap 606 remains on the staple cartridge 100 to be removed when the surgical instrument is ready to be used for the stapling procedure. First breakaway portion 602 or second breakaway portion 504 includes male connector 506 that engages with female connector 508 on the opposite breakaway portion. The male connector 506 in the example embodiment includes two tabs that can be compressed inwardly to snap into the respective female connector 508. As will be appreciated, the breakaway portions can include more than one male connector 506 and female connector 508, spaced evenly along retainer (e.g., along length 462 of adjunct shelf 460). Folding first retainer tab 406 and second retainer tab 408 into their folded states can provide the force to separate the respective male connectors 506 and female connectors 508 to remove the portions from adjunct cap 606.

Referring to FIG. 6E, adjunct cap 606 shown in the figure also includes two retention tabs 414 extending from retainer 400. Retention tabs 414 are outwardly deflectable upon seating into first jaw frame 204. Retention tabs 414 can keep adjunct cap 606 connected to staple cartridge 100 until the cartridge/cap is inserted into first jaw frame 204. For instance, retention tabs 414 can hook onto cartridge rails 118 of staple cartridge 100. Upon insertion into first jaw frame 204, channel rails 208 of first jaw frame 204 will slip between retention tabs 414 and staple cartridge 100 to deflect them outwardly, thereby releasing them from cartridge rails 118 such that adjunct cap 606 can be removed from staple cartridge 100. Adjunct cap 606 can also include sled positioning rib 608 that extends into sled groove 304 (see FIG. 2C for sled groove 304), for adjuncts 300 that include said sled groove 304. Sled positioning rib 608 can ensure separation of adjunct first side 306 and adjunct second side 308 (FIG. 2C) during shipment. Further, sled positioning rib 608 can prevent a sled (not shown in figures) positioned inside staple cartridge 100 from moving distally before the surgical instrument 200 fires said sled during the stapling procedure.

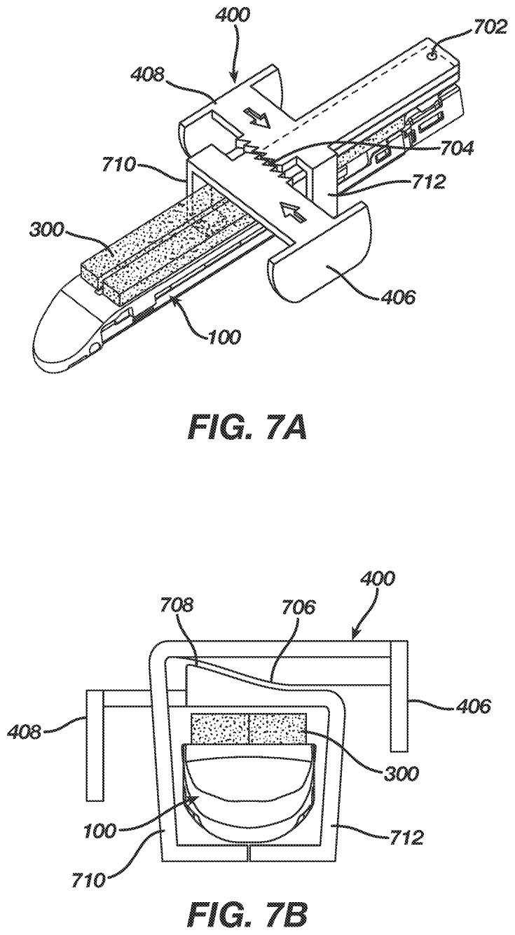

Referring now to FIGS. 7A and 7C, the example shows a staple cartridge and retainer system that can protect an adjunct during shipment of the staple cartridge and also provide a compressive force to the adjunct by moving respective retainer tabs inwardly. In this implementation, first retainer tab 406 and second retainer tab 408 are pivotably connected to each other, for example via pivot 702. As such, a user (e.g., physician or other surgical personnel) can provide the compressive force to adjunct 300 by moving first retainer tab 406 and second retainer tab 408 toward each other. First retainer tab 406 includes first ramp 706, and second retainer tab 408 includes second ramp 708. By actuating the first retainer tab 406 and second retainer tab 408 (e.g., by moving them toward each other), first ramp 706 and second ramp 708 slide with respect to each other to provide uniform compression downward onto adjunct 300. Although FIG. 7A shows retainer 400 extension only partially along the length (see length 350 in FIG. 2C), it is contemplated that retainer 400 can extend the entire length of adjunct 300. Further, first ramp 706 and second ramp 708 can extend the length of retainer 400 so that when first retainer tab 406 and second retainer tab 408 are actuated toward each other, the compressive force is provided across the entire length of retainer 400.

First retainer tab 406 and second retainer tab 408 each comprise sets of ratcheting teeth 704 engageable with each other. Ratcheting teeth 704 enable first retainer tab 406 and second retainer tab 408 to only move toward each other and not away from each other. Further, retainer 400 comprises first lateral hook 710 extending at least partially around elongate body 120 of staple cartridge 100 and opposite first retainer tab 406. Retainer 400 comprises second lateral hook 712 extending at least partially around the elongate body 120 of staple cartridge 100 and opposite second retainer tab 408. When first retainer tab 406 and second retainer tab 408 are actuated toward each other, first lateral hook 710 and second lateral hook 712 move to an expanded position wherein they do not retain staple cartridge 100, allowing staple cartridge 100 to be removed between the respective lateral hook 710, 712 (expanded position of first lateral hook 710 and second lateral hook 712 not shown in FIGS. 7A and 7B). FIG. 7B is an end view of retainer 400 that includes pivotable first retainer tab 406 and second retainer tab 408.

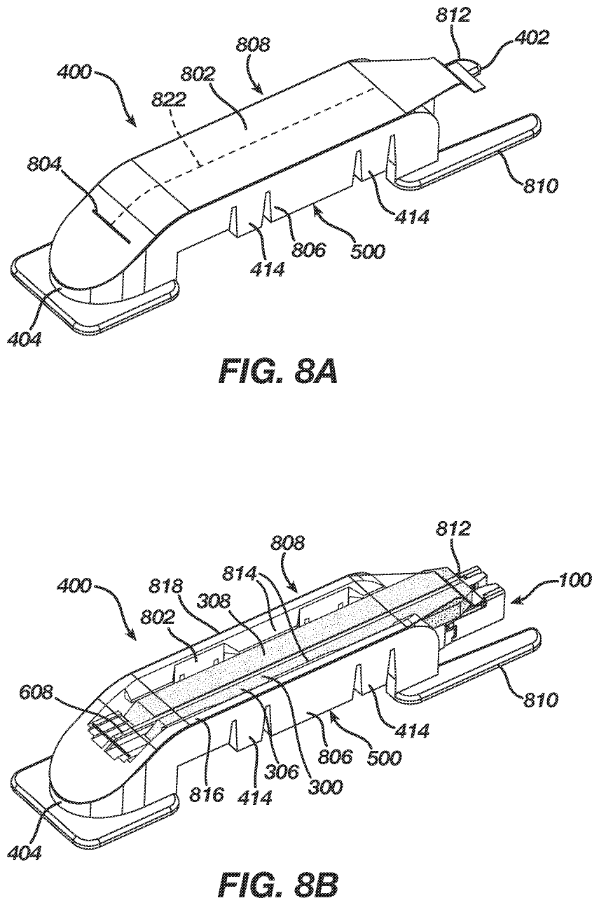

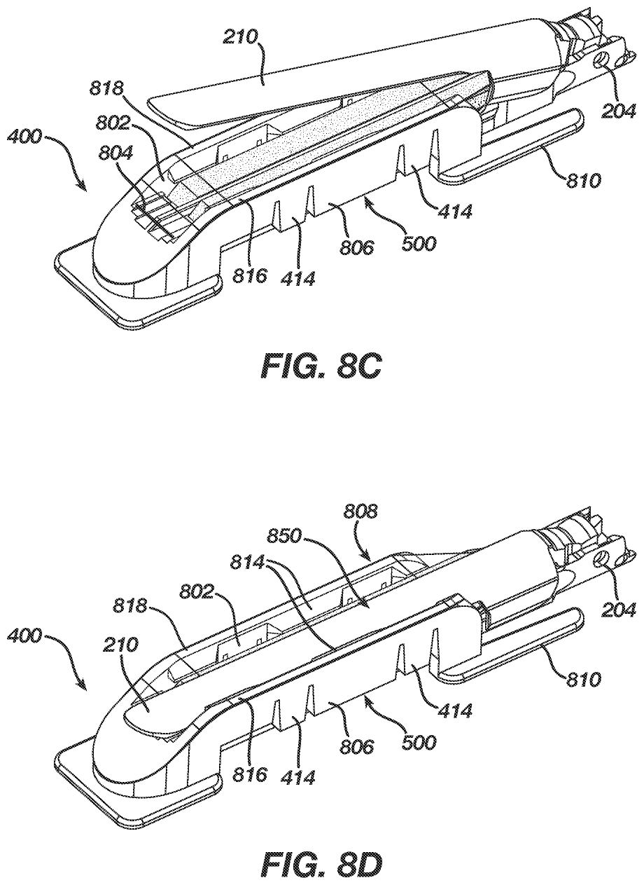

Turning now to FIGS. 8A-8D, the example retainer 400 shown therein includes a film 802 that can be torn by closing of anvil 210. Retainer body 500 includes first lateral wall 806 and second lateral wall 808 having first lateral wall lip 816 and second lateral wall lip 818, respectively. Film 802 extends between first lateral wall 806 and second lateral wall 808, and film 802 covers and protects the adjunct 300 therein, i.e., film 802 is disposed along a side of implantable adjunct 300 opposite deck 108. Film 802 can be connected to first lateral wall lip 816 and second lateral wall lip 818 by adhesives, heat staking, or any other method of adhering or welding two plastics together. In certain implementations, first lateral wall 806 and second lateral wall 808 can be manufactured such that they are biased outwardly, such that they diverge from each other in a direction away from distal end 404 of retainer 400. As such, film 802 can ensure first lateral wall 806 and second lateral wall 808 are maintained in their parallel configurations (see FIG. 8A) until film 802 is torn. Once film 802 is torn, first lateral wall 806 and second lateral wall 808 can separate from each other to enable retainer 400 to be easily removed from staple cartridge 100 and/or first jaw frame 204.

Referring now specifically to film 802, the film is set at a height higher than implantable adjunct 300 such that retainer 400 is placed in first jaw frame 204 along with staple cartridge 100 (FIG. 8B shows retainer 400 attached to staple cartridge 100). Once retainer 400 is in first jaw frame 204, anvil 210 is closed such that the anvil contacts film 802. While closing, anvil 210 first contacts film 802 proximally and can tear film 802 layer as it closes on implantable adjunct 300. The tearing away of film 802 starts proximally and continues distally (i.e., towards distal end 404) until anvil 210 is pressing down on implantable adjunct 300. This closure of anvil 210 onto implantable adjunct 300 provides the compression to enhance the attachment material 302 adhesive effectiveness. As will be appreciated, tearing of the film can be understood to mean the film can be made of a plastic material that has sufficiently low elasticity to ensure that it is torn by closing of anvil 210 without merely being stretched, but low enough in toughness that the film tears via actuation of anvil 210 between first lateral wall 806 and second lateral wall 808. In these implementations, film 802 can also include a longitudinal sore line 822 along a length of film 802 to assist in tearing the film down the middle. In other examples, “tearing” of the film can include ripping or separating film 802 from one side of cartridge 100, as in ripping or separating film 802 from either first lateral wall lip 816 or second lateral wall lip 818. As anvil 210 closes and contacts film 802, therefore, the pressure can separate at least one side of film 802 from the respective lateral wall lip so that adjunct 300 can be compressed.

As shown, for example, in FIG. 8A, some implementations of retainer 400 can include score line 804 proximate distal end 404 of retainer 400. Score line 804 provides a pre-torn section of film 802 at the location where anvil 210 will rest when in a fully closed position. A portion of film 802 is connected to (e.g., adhered, head staked, etc.) to retainer 400 distal to score line 804. When anvil 210 tears the film while closing, only the portion of film 802 proximal to the score line 804 tears, yet film 802 will remain connected to retainer 400 at first lateral wall lip 816, second lateral wall lip 818, and the area of retainer 400 distal to score line 804. When retainer 400 is removed from staple cartridge 100, therefore, film 802 will also be removed so that staple cartridge 100 is ready for the stapling procedure.

FIG. 8C shows a retainer/cartridge loaded into first jaw frame 204 but before anvil 810 has torn film 802. As shown, retainer 400 can include extensions, i.e., first extension 810 and second extension 812. These extensions 810, 812 can help to align staple cartridge 100 as it is positioned to be inserted into first jaw frame 204. FIG. 8D shows a retainer/cartridge loaded into first jaw frame 204 after anvil 810 has torn film 802. As shown, retainer 400 can include anvil grips 814 positioned on the inside surfaces of first lateral wall 806 and second lateral wall 808. Anvil grips 814 can be lips that are sloped on the top and flat on their bottoms so as to engage with anvil 210 as it closes. To illustrate, as anvil 210 closes from its open configuration (FIG. 8C) to its closed configuration (FIG. 8D), anvil 210 will slip between said anvil grips 814; when anvil 210 is reopened, retainer 400 will remain attached to anvil 210 via said anvil grips 814 so that it can be removed from end effector 202 (see FIG. 3).

As shown in FIG. 8B in particular, retainer 400 can include sled positioning rib 608 that extends into sled groove 304 (see FIG. 2C for sled groove 304), for adjuncts 300 that include said sled groove 304. Sled positioning rib 608 can ensure separation of adjunct first side 306 and adjunct second side 308 (FIG. 2C) during shipment. Further, sled positioning rib 608 can prevent a sled (not shown in figures) positioned inside staple cartridge 100 from moving distally before the surgical instrument 200 fires said sled during the stapling procedure. Sled positioning rib 608 can be attached to the retainer body 500 of retainer 400 proximate score line 804.

Retainer 400 shown in FIGS. 8A-8D also includes two retention tabs 414 extending from retainer 400. Retention tabs 414 are outwardly deflectable upon seating into first jaw frame 204. Retention tabs 414 can keep retainer 400 connected to staple cartridge 100 until the cartridge/retainer is inserted into first jaw frame 204. For instance, retention tabs 414 can hook onto cartridge rails 118 of staple cartridge 100 (see FIGS. 2A and 2B). Upon insertion into first jaw frame 204, channel rails 208 of first jaw frame 204 will slip between retention tabs 414 and staple cartridge 100 to deflect them outwardly, thereby releasing them from cartridge rails 118 such that retainer 400 can be removed from staple cartridge 100. FIG. 5F, although depicting a different embodiment of retainer 400, provides an illustration of retention tabs 414 hooked to cartridge rails 118 before being deflected by channel rails 208 of first jaw frame 204.

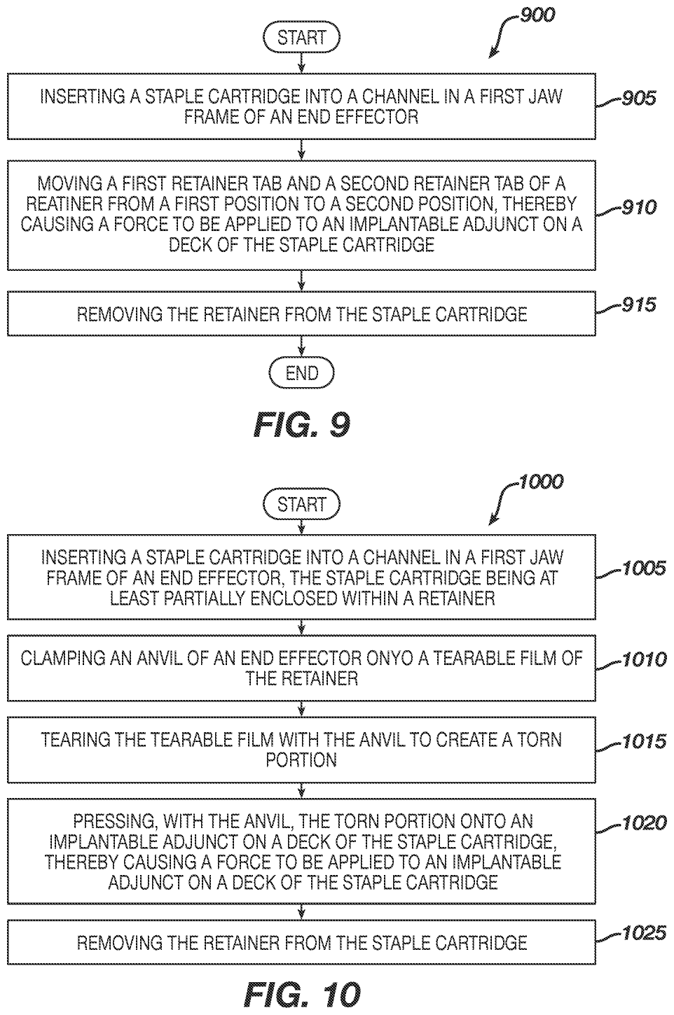

FIG. 9 is a flowchart for an example method 900 of loading a staple cartridge 100 onto an end effector 202, according to aspects of the present disclosure. Method 900 can be performed to load any of the retainers 400 shown in FIGS. 4A-7B into an end effector 202 (see FIG. 3 for end effector 202). Method 900 includes inserting 905 staple cartridge 100 into channel 206 of first jaw frame 204 of end effector 202. As described herein, retainer 400 can be attached to staple cartridge 100 when inserted into first jaw frame 204.

Method 900 includes moving 910 first retainer tab 406 and second retainer tab 408 of retainer 400 from a first position to a second position, thereby causing a force to be applied to implantable adjunct 300 on deck 108 of staple cartridge 100. As described above, said second position is where at least a portion of first retainer tab 406 and second retainer tab 408 are closer to elongate body 120 of staple cartridge 100 than in said first position. The force applied by this moving 910 step increases an adhesion of implantable adjunct 300 to deck 108 by providing pressure to attachment material 302 disposed between implantable adjunct 300 and deck 108.

In some implementations, the moving 910 step can include folding the respective retainer tabs downwardly to a position below first jaw frame 204. Further, the step can also include separating retainer 400 into first breakaway portion 502 and second breakaway portion 504 responsive to moving first retainer tab 406 and second retainer tab 408 into the second position (i.e., for the embodiments in FIGS. 5A-6E that include breakaway portions). Method 900 includes removing 915 retainer 400 from staple cartridge 100 so that end effector 202 can be inserted into the patient for the stapling procedure. Method 900 can end after the removing 915 step, or additional steps can be performed according to the embodiments described herein.

FIG. 10 is a flowchart for an example method 1000 of loading a staple cartridge 100 onto an end effector 202, according to aspects of the present disclosure. Method 1000 can be performed to load the retainer 400 shown in FIGS. 8A-8D into an end effector 202 (see FIG. 3 for end effector 202). Method 1000 includes inserting 1005 staple cartridge 100 into channel 206 of first jaw frame 204 of an end effector 202. The staple cartridge 100 is at least partially enclosed within retainer 400. Method 1000 includes clamping 1010 anvil 210 of end effector 202 onto film 802 of retainer 400.

Method 1000 includes tearing 1015 film 802 with anvil 210 to create a torn portion 850 (see FIG. 8D). Method 1000 includes pressing 1020, with anvil 210, the torn portion 850 of film 802 onto implantable adjunct 300 on deck 108 of staple cartridge 100. The pressing 1020 step can increase an adhesion of implantable adjunct 300 to deck 108 by providing pressure to attachment material 302 disposed between implantable adjunct 300 and deck 108. A first portion of film 802 distal to a score line (e.g., score line 804) can remain adhered to retainer 400 after tearing film 802 with anvil 210.

Method 1000 includes removing 1025 retainer 400 from staple cartridge 100. Method 1000 can end after the removing 1025 step, or additional steps can be performed according to the embodiments described herein. For example, method 1000 can include deflecting one or more retention tabs 414 extending from retainer 400 outwardly upon engagement with channel rails 208 of channel 206. Method 1000 can include engaging anvil 210 with anvil grips 814 on retainer 400, raising anvil 210, and lifting retainer 400—while the anvil 210 is raising—via anvil grips 814.

Examples of the present disclosure can be implemented by any of the following numbered clauses:

Clause 1: A staple cartridge (100), comprising: an elongate body (120), the elongate body (120) comprising a deck (108), the elongate body (120) defining a plurality of staple pockets (110), each of the staple pockets (110) accessible via an opening (112) in the deck (108); an implantable adjunct (300) removably secured to the deck (108); and a retainer (400) removably securable to the elongate body (120), the retainer (400) comprising: a first retainer tab (406) extending laterally with respect to a longitudinal axis (450) of the retainer (400); and a second retainer tab (408) extending laterally with respect to the longitudinal axis (450) of the retainer (400), the first retainer tab (406) and the second retainer tab (408) movable from a first position to a second position, in the second position the retainer (400) providing a force to compress the implantable adjunct (300) against the deck (108) of the elongate body (120), and at least a portion of the first retainer tab (406) and the second retainer tab (408) being closer to the elongate body (120) in the second position than in the first position.

Clause 2: The staple cartridge (100) of Clause 1, wherein the retainer (400) comprises a retainer body (500) extending along the longitudinal axis (450), and wherein the first retainer tab (406) and the second retainer tab (408) are connected to the retainer body (500) respectively via a first living hinge (410) and a second living hinge (412).

Clause 3: The staple cartridge (100) of Clause 1 or 2, wherein the first retainer tab (406) and the second retainer tab (408) comprise an angled profile such that a portion of the respective tabs (406, 408) is positioned on a side of the elongate body (120) opposite the implantable adjunct (300).

Clause 4: The staple cartridge (100) of any of Clauses 1 to 3, wherein the retainer (400) comprises a first breakaway portion (502) and a second breakaway portion (504) that are separable along the longitudinal axis (450) of the retainer (400).

Clause 5: The staple cartridge (100) of Clause 4, wherein one of the first breakaway portion (502) and the second breakaway portion (504) comprises a female connector (508), and wherein the other of the second breakaway portion (504) and the first breakaway portion (502) comprises a male connector (506) engageable with the female connector (508).

Clause 6: The staple cartridge (100) of Clause 4 or 5, wherein the first breakaway portion (502) and the second breakaway portion (504) are separable in response to the first retainer tab (406) and the second retainer tab (408) moving to the second position.

Clause 7: The staple cartridge (100) of any of Clauses 1 to 6 further comprising one or more retention tabs (414) extending from the retainer (400), the retention tabs (414) being outwardly deflectable.

Clause 8: The staple cartridge (100) of any of Clauses 1 to 7 further comprising an adjunct cap (606) disposed between the implantable adjunct (300) and the retainer (400).

Clause 9: The staple cartridge (100) of Clause 8, wherein the adjunct cap (606) further comprises at least one retention tab (414) extending therefrom, the at least one retention tab (414) being outwardly deflectable upon engagement with at least one channel rail (208) of an end effector (202).

Clause 10: The staple cartridge (100) of Clause 8 or 9, wherein the implantable adjunct (300) comprises a sled groove (304) separating the implantable adjunct (300) into a first side (306) and a second side (308), and wherein the adjunct cap (606) comprises a rib (608) configured to extend into the sled groove (304).

Clause 11: The staple cartridge (100) of any of Clauses 1 to 10, wherein the first retainer tab (406) and the second retainer tab (408) are positioned centrally along a length (462) of an adjunct shelf (460) to provide uniform compression along a length (350) of the implantable adjunct (300).

Clause 12: The staple cartridge (100) of Clause 1, wherein: the first retainer tab (406) and the second retainer tab (408) are pivotably connected to each other; the first retainer tab (406) comprises a first ramp (706); the second retainer tab (408) comprises a second ramp (708) engageable with the first ramp (706); and moving from the first position to the second position causes the first retainer tab (406) and the second retainer tab (408) to slide with respect to each other and provide uniform compression along a length (350) of the implantable adjunct (300) against the deck (108) of the elongate body (120).

Clause 13: The staple cartridge (100) of Clause 12, wherein the first retainer tab (406) and the second retainer tab (408) each comprise sets of ratcheting teeth (704) engageable with each other.

Clause 14: The staple cartridge (100) of Clauses 12 or 13, wherein the retainer (400) comprises a first lateral hook (710) extending at least partially around the elongate body (120) and opposite the first retainer tab (406), and wherein the retainer (400) comprises a second lateral hook (712) extending at least partially around the elongate body (120) and opposite the second retainer tab (408).

Clause 15: The staple cartridge (100) of Clause 14, wherein the first lateral hook (710) and the second lateral hook (712) each have ends that are separable from each other in response to the first retainer tab (406) and the second retainer tab (408) moving from the first position to the second position.

Clause 16: A method of loading a staple cartridge onto an end effector, the method comprising the steps of: inserting a staple cartridge (100) into a channel (206) of a first jaw frame (204) of an end effector (202); moving a first retainer tab (406) and a second retainer tab (408) of a retainer (400) from a first position to a second position, thereby causing a force to be applied to an implantable adjunct (300) on a deck (108) of the staple cartridge (100), the force increasing an adhesion of the implantable adjunct (300) to the deck (108) by providing pressure to an attachment material (302) disposed between the implantable adjunct (300) and the deck (108); and removing the retainer (400) from the staple cartridge (100).

Clause 17: The method of Clause 16, wherein the moving the first retainer tab (406) and the second retainer tab (408) step comprises folding the respective retainer tabs downwardly to a position below the first jaw frame (204).

Clause 18: The method of Clause 16 or 17 further comprising the step of: separating the retainer (400) into a first breakaway portion (502) and a second breakaway portion (504) responsive to moving the first retainer tab (406) and the second retainer tab (408) into the second position.

Clause 19: The method of any of Clauses 16 to 18 further comprising the step of: removing an adjunct cap (606) from the staple cartridge (100) to expose the implantable adjunct (300).

Clause 20: The method of Clause 16, wherein the moving the first retainer tab (406) and the second retainer tab (408) step comprises squeezing the respective tabs inwardly to cause a first ramp (706) the first retainer tab (406) to engage with a second ramp (708) of the second retainer tab (408) to engage and provide the downward force.

Clause 21: A staple cartridge (100), comprising: an elongate body (120), the elongate body (120) comprising a deck (108), the elongate body (120) defining a plurality of staple pockets (110), each of the staple pockets (110) being accessible via an opening (112) in the deck (108); an implantable adjunct (300) removably secured to the deck (108); and a retainer (400) removably securable to the elongate body (120), the retainer (400) comprising: a retainer body (500) comprising a first lateral wall (806) and a second lateral wall (808); and a film (802) extending between the first lateral wall (806) and the second lateral wall (808) and disposed along a side of the implantable adjunct (300) opposite the deck (108).

Clause 22: The staple cartridge (100) of Clause 21, wherein the film (802) are attached to the retainer body (400) at a first lateral wall lip (816) and a second lateral wall lip (818).

Clause 23: The staple cartridge (100) of Clause 21 or 22, wherein the film (802) comprises a score line (804) proximate a distal end (404) of the retainer (400).

Clause 24: The staple cartridge (100) of Clause 23, wherein the film (802) is attached to the retainer body (500) distal to the score line (804).

Clause 25: The staple cartridge (100) of Clause 23 or 24, wherein a first portion of the film (802) distal to the score line (804) is adhered sufficiently to maintain being attached to the distal end (404) of the retainer (400) after a second portion of the film (802) proximal to the score line (804) is torn by an anvil (210) of an end effector (202).

Clause 26: The staple cartridge (100) of any of Clauses 21 to 25 further comprising one or more retention tabs (414) extending from the retainer body (500), the retention tabs (414) being outwardly deflectable.

Clause 27: The staple cartridge (100) of any of Clauses 21 to 26 further comprising a plurality of anvil grips (814) engageable with an anvil (210) of an end effector (202).

Clause 28: The staple cartridge (100) of any of Clauses 21 to 27, wherein the implantable adjunct (300) comprises a sled groove (304) separating the implantable adjunct (300) into a first side (306) and a second side (308), and wherein the adjunct cap (606) comprises a rib (608) configured to extend into the sled groove (304).

Clause 29: The staple cartridge (100) of Clause 28, wherein the film (802) comprises a score line (804) proximate a distal end (404) of the retainer (400), and wherein the rib (608) is attached to the retainer body (500) proximate the score line (804) proximate a distal end (404) of the retainer (400).

Clause 30: A method of loading a staple cartridge onto an end effector, the method comprising the steps of: inserting a staple cartridge (100) into a channel (206) of a first jaw frame (204) of an end effector (202), the staple cartridge (100) being at least partially enclosed within a retainer (400); clamping an anvil (210) of an end effector (202) onto a film (802) of the retainer (400); tearing the film (802) with the anvil (210) to create a torn portion (850); pressing, with the anvil (210), the torn portion (850) onto an implantable adjunct (300) on a deck (108) of the staple cartridge (100); and removing the retainer (400) from the staple cartridge (100).

Clause 31: The method of Clause 30 further comprising the step of: deflecting one or more retention tabs (414) extending from the retainer (400) outwardly upon engagement with channel rails (208) of the channel (206).

Clause 32: The method of Clause 30 or 31 further comprising the steps of: engaging the anvil (210) with anvil grips (814) on the retainer (400); raising the anvil (210); and lifting the retainer (400), while the anvil (210) is raising, via the anvil grips (814).

Clause 33: The method of any of Clauses 30 to 32, wherein a first portion of the film (802) distal to a score line (804) remains adhered to the retainer (400) after tearing the film (802) with the anvil (210).

Clause 34. The method of any of Clauses 30 to 33, wherein the pressing the torn portion (850) onto the implantable adjunct (300) step further comprises increasing an adhesion of the implantable adjunct (300) to the deck (108) by providing pressure to an attachment material (302) disposed between the implantable adjunct (300) and the deck (108).

The invention is not necessarily limited to the examples described, which can be varied in construction and detail. The terms “distal” and “proximal” are used throughout the preceding description and are meant to refer to a positions and directions relative to the handle of surgical instrument 200. As such, “distal” or distally” refer to a position distant to or a direction away from the handle of surgical instrument 200 (i.e., a direction toward a patient). Similarly, “proximal” or “proximally” refer to a position near or a direction towards the robotic arm. Furthermore, the singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 80.1% to 99.9%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

1-15. (canceled)

16. A staple cartridge, comprising:
an elongate body, the elongate body comprising a deck, the elongate body defining a plurality of staple pockets, each of the staple pockets accessible via an opening in the deck;
an implantable adjunct removably secured to the deck; and
a retainer removably securable to the elongate body, the retainer comprising:
a first retainer tab extending laterally with respect to a longitudinal axis of the retainer; and
a second retainer tab extending laterally with respect to the longitudinal axis of the retainer,
the first retainer tab and the second retainer tab movable from a first position to a second position, in the second position the retainer providing a force to compress the implantable adjunct against the deck of the elongate body, and at least a portion of the first retainer tab and the second retainer tab being closer to the elongate body in the second position than in the first position.

17. The staple cartridge of claim 16, wherein the retainer comprises a retainer body extending along the longitudinal axis, and wherein the first retainer tab and the second retainer tab are connected to the retainer body respectively via a first living hinge and a second living hinge.

18. The staple cartridge of claim 16, wherein the first retainer tab and the second retainer tab comprise an angled profile such that a portion of the respective tabs is positioned on a side of the elongate body opposite the implantable adjunct.

19. The staple cartridge of claim 16, wherein the retainer comprises a first breakaway portion and a second breakaway portion that are separable along the longitudinal axis of the retainer.

20. The staple cartridge of claim 19, wherein one of the first breakaway portion and the second breakaway portion comprises a female connector, and wherein the other of the second breakaway portion and the first breakaway portion comprises a male connector engageable with the female connector.

21. The staple cartridge of claim 19, wherein the first breakaway portion and the second breakaway portion are separable in response to the first retainer tab and the second retainer tab moving to the second position.

22. The staple cartridge of claim 16 further comprising one or more retention tabs extending from the retainer, the retention tabs being outwardly deflectable.

23. The staple cartridge of claim 16 further comprising an adjunct cap disposed between the implantable adjunct and the retainer.

24. The staple cartridge of claim 23, wherein the adjunct cap further comprises at least one retention tab extending therefrom, the at least one retention tab being outwardly deflectable upon engagement with at least one channel rail of an end effector.

25. The staple cartridge of claim 23, wherein the implantable adjunct comprises a sled groove separating the implantable adjunct into a first side and a second side, and wherein the adjunct cap comprises a rib configured to extend into the sled groove.

26. The staple cartridge of claim 16, wherein the first retainer tab and the second retainer tab are positioned centrally along a length of an adjunct shelf to provide uniform compression along a length of the implantable adjunct.

27. The staple cartridge of claim 16, wherein:
the first retainer tab and the second retainer tab are hingeably connected to each other;
the first retainer tab comprises a first ramp;
the second retainer tab comprises a second ramp engageable with the first ramp; and
moving from the first position to the second position causes the first retainer tab and the second retainer tab to slide with respect to each other and provide uniform compression along a length of the implantable adjunct against the deck of the elongate body.

28. The staple cartridge of claim 27, wherein the first retainer tab and the second retainer tab each comprise sets of ratcheting teeth engageable with each other.

29. The staple cartridge of claim 27, wherein the retainer comprises a first lateral hook extending at least partially around the elongate body and opposite the first retainer tab, and wherein the retainer comprises a second lateral hook extending at least partially around the elongate body and opposite the second retainer tab.

30. The staple cartridge of claim 29, wherein the first lateral hook and the second lateral hook each have ends that are separable from each other in response to the first retainer tab and the second retainer tab moving from the first position to the second position.

31. A method of loading a staple cartridge onto an end effector, the method comprising the steps of:
inserting a staple cartridge into a channel of a first jaw frame of an end effector;
moving a first retainer tab and a second retainer tab of a retainer from a first position to a second position, thereby causing a force to be applied to an implantable adjunct on a deck of the staple cartridge, the force increasing an adhesion of the implantable adjunct to the deck by providing pressure to a buttress attachment material disposed between the implantable adjunct and the deck; and
removing the retainer from the staple cartridge.

32. The method of claim 31, wherein the moving the first retainer tab and the second retainer tab step comprises folding the respective retainer tabs downwardly to a position below the first jaw frame.

33. The method of claim 31 further comprising the step of:
separating the retainer into a first breakaway portion and a second breakaway portion responsive to moving the first retainer tab and the second retainer tab into the second position.

34. The method of claim 31 further comprising the step of:
removing an adjunct cap from the staple cartridge to expose the implantable adjunct.

35. The method of claim 31, wherein the moving the first retainer tab and the second retainer tab step comprises squeezing the respective tabs inwardly to cause a first ramp the first retainer tab to engage with a second ramp of the second retainer tab to engage and provide the downward force.
